SEQUENCE LISTING (17 sequences)

SEQ ID NO: 1 - protein, 9 aa, Escherichia coli
VERETAQIL

SEQ ID NO: 2 - protein, 12 aa, Escherichia coli
KKSSTTSAIGIL

SEQ ID NO: 3 - protein, 14 aa, Escherichia coli
RQRFSGISPPVSDP

SEQ ID NO: 4 - protein, 24 aa, Escherichia coli
SAAEADRNDCGNPVQREDRRGSDP

SEQ ID NO: 5 - protein, 26 aa, Escherichia coli
HNPYWRLTESSDVLRFSTTETTEPDP

SEQ ID NO: 6 - protein, 29 aa, Escherichia coli
GVAPCLRWKRCTRTRRPSGARTSGWTRSV

SEQ ID NO: 7 - protein, 34 aa, Escherichia coli
RGAPCRPSGRAVYERQTCARPFQPEHVQSRPYDP

SEQ ID NO: 8 - protein, 12 aa, Escherichia coli
PNGAAPLKGRSV

SEQ ID NO: 9 - protein, 16 aa, Escherichia coli
GSRSRGPRRPGSPGSV

SEQ ID NO: 10 - protein, 23 aa, Escherichia coli
PRCPRRSRASHHEEVVVAASGDP

SEQ ID NO: 11 - protein, 26 aa, Escherichia coli
HPLPAGSPGAFSSETAPRIARRRSRS

SEQ ID NO: 12 - protein, 29 aa, Escherichia coli
WTRSVGVAPCLRWKRCTRTRRPSGARTSG

SEQ ID NO: 13 - protein, 36 aa, Escherichia coli
KESAKTAGNNERRTGRPTSGAFPLRAGISASRGRSV

SEQ ID NO: 14 - protein, 36 aa, Escherichia coli
GQKTAPPPWPGPPFCGRGSPTRPAPRGKTRSARRSV

SEQ ID NO: 15 - protein, 48 aa, Escherichia coli
PSSRSFPQGLSGARMDGMGTLTRYLEEAMARARYELIADEEPYYGEIL

SEQ ID NO: 16 - protein, 48 aa, Escherichia coli
PSGGGMSRTKRSRTSCTPTPVFAEQRTASVASMPTISSISSATRSGSV

SEQ ID NO: 17 - protein, 52 aa, Escherichia coli
PRARGCTRRRCRGGSRRSGRGRRPPVPYRAPCPQCPRGPASWPRSPRLCRDP

(19) United States
(12) Patent Application Publication
(10) Pub. No.: US 2003/0049686 A1
(43) Pub. Date: Mar. 13, 2003

(54) Method for manufacturing mutnat library of proteins with various sizes and sequences

(75) Inventors: Hak-Sung Kim, Taejon (KR); Geun-Joong Kim, Suwon (KR); Young-Hoon Cheon, Taejon (KR); Dong-Eun Lee, Taejon (KR)

Correspondence Address: DARBY & DARBY P.C., 805 Third Avenue, New York, NY, 10022, US

(73) Assignee: Korea Advanced Institute of Science and Technology

(21) Appl. No.: 10/112,455

(22) Filed: Mar. 29, 2002

(30) Foreign Application Priority Data
Sep. 10, 2001 (KR) 10-2001-0055394

(51) Int. Cl. C12Q 1/68; G01N 33/53; C12P 19/34; C12P 21/02; C12N 5/06; C07K 14/00

(52) U.S. Cl. 435/7.1; 435/69.1; 435/91.2; 435/252.3; 530/350

(57) ABSTRACT

The present invention relates to a method for manufacturing a mutant library of proteins with various sizes and sequences from a parental protein, microorganisms transformed with plasmids containing recombinant DNAs prepared by the insertion of a genomic DNA fragment into a defective template, a process for preparing proteins with different sizes and sequences from the parental protein which comprises the steps of culturing the transformed microorganisms and obtaining desired proteins from the culture, and proteins prepared by the said process. In accordance with the invention, a mutant library of proteins with various sizes and sequences can be manufactured from a parental protein in an efficient and simple manner, by constructing a library of microorganisms transformed with recombinant plasmids containing E. coli genomic DNA fragments inserted into defective genes and selecting clones expressing proteins with restored functions or modified characteristics.

### FIELD OF THE INVENTION

The present invention relates to a method for manufacturing a mutant library of proteins with various sizes and sequences from a parental protein, more specifically, to a method for manufacturing a mutant library of proteins with various sizes and sequences from a parental protein, microorganisms transformed with plasmids containing recombinant DNAs prepared by the insertion of a genomic DNA fragment into a defective template, a process for preparing proteins with different sizes and sequences from the parental protein which comprises the steps of culturing the transformed microorganisms and obtaining desired proteins from the culture, and proteins prepared by the said process.

### BACKGROUND OF THE INVENTION

Proteins have been widely utilized in pharmaceutical, therapeutic and industrial fields, and studies on the modification of their functions and characteristics have been continuously made in the art. The most common method is that a mutant library is obtained through an artificial modification into a gene coding for a protein and proteins with changed functions are prepared therefrom. Thus, the production of proteins with changed functions or characteristics is largely dependent on the manufacture of a mutant library of proteins.

As the methods for manufacturing a mutant library of proteins, random mutations into the coding region of a protein and substitution, deletion, or addition of a specific amino acid into a parental protein, based on the protein structure, have been chiefly employed in the art, and an error-prone PCR has been employed as a more efficient method. Recently, DNA shuffling method has developed by Maxygen Co. (USA) and enjoyed the distinction as a method for manufacturing a various mutant library of proteins, and there are many reports on the application of this method. However, this method is proven to be less satisfactory in a sense that: it produces mutant libraries of proteins without changing the size of genes coding for parental proteins by way of a point mutation or a recombination based on sequence homology, which naturally limits the production of more various proteins.

It has been well known that various proteins aroused during a natural evolution process are produced not only through changes in the nucleotide sequence but also through a complex process such as substitution, deletion, or addition of a gene fragment with random size or nucleotide sequence. Based on this knowledge, a method for manufacturing proteins with different sizes and sequences from their parental proteins has been developed by the addition of oligonucleotides of random sizes and sequences into a gene corresponding to the carboxy terminus of a protein. In addition, other methods employing sub-domain swapping, domain or module grafting, and recombination independent on the sequence homology have been reported in the art. However, all of these prior methods do not contribute to the diversification of a mutant library.

Under the circumstances, there are strong reasons for exploring and developing an efficient, simple method for manufacturing a diversified mutant library of proteins with various sizes and sequences from a parental protein along with various functions and characteristics.

### SUMMARY OF THE INVENTION

The present inventors have made an effort to develop a method for manufacturing a mutant library of proteins with various sizes and sequences from a parental protein, constructed a library of microorganisms transformed with a plasmid containing a recombinant DNA prepared by the insertion of a genomic DNA fragment into a defective template, and found that a mutant library of proteins with various sizes and sequences from a parental protein can be produced therefrom.

A primary object of the invention is, therefore, to provide a method for manufacturing a mutant library of proteins with various sizes and sequences from a parental protein.

The other object of the invention is to provide a microorganism transformed with a plasmid containing a recombinant gene prepared by the insertion of a genomic DNA fragment into a defective template.

Another object of the invention is to provide proteins selected from the proteins expressed by the microorganisms.

### BRIEF DESCRIPTION OF DRAWINGS

The above and the other objects and features of the present invention will become apparent from the following descriptions given in conjunction with the accompanying drawings, in which:

FIG. 1 is a schematic diagram showing a method for manufacturing a mutant library of proteins with various sizes and sequences from a parental protein of the present invention.

FIG. 2 is a photograph showing the results of agarose gel electrophoresis of genes of clones randomly selected from a library of microorganisms constructed with a defective template, GFP∇176(+2).

FIGS. 3a and 3b represent amino acid sequences deduced from E. coli genomic DNA fragment inserted into defective templates, GFP∇176(+2) and GFP∇172-3/176(+2) and ORF sizes of GFP∇176(+2) and GFP∇172-3/176(+2), respectively.

### DETAILED DESCRIPTION OF THE INVENTION

The method for manufacturing a mutant library of proteins with various sizes and sequences from a parental protein of the present invention employs a defective template obtained by an artificial modification. The artificial modification is made into a specific site whose mutation is critical for normal functioning of a protein, thereby destroying the function of the protein. The selection of the site is made based on the protein structure and sequence homology to related proteins. The defective template prepared by a mutation to a critical site of gene coding for a protein, makes easy the selection of proteins with changed characteristics or restored functions, which are expressed from a library of microorganisms constructed, and the control of a modification restoring the function only at the desired site. This usefulness of the defective template is resulted from the facts that: a modification has to be occurred to generate a protein with a new sequence and size before the restoration of destroyed function; this modification is induced by the insertion of a random oligonucleotide or a genomic DNA fragment prepared with the treatment of restriction enzymes; and, the site for the insertion can be artificially selected.

The present invention is illustrated in more detail as followings.

The method for manufacturing a mutant library of proteins with various sizes and sequences from a parental protein comprises the steps of: artificial modification into a gene coding for a parental protein to give a defective template; construction of a library of microorganisms transformed with plasmids containing recombinant DNAs prepared by the insertion of a random oligonucleotide or a restriction enzyme-treated genomic DNA fragment to the defective template; and, selection of proteins having restored function or changed characteristics from proteins expressed by the library of microorganisms transformed with plasmids.

In carrying out the method, the artificial mutation includes deletion of nucleotides coding for several amino acids or a domain at a specific site of a gene coding for a parental protein, frame shift and combination thereof. The oligonucleotides to be inserted to the specific site of the defective template is randomly synthesized not by the addition of dATP, dGTP, dTTP, and dCTP in an ordered fashion but by the addition of all 4 or less than 4 nucleotides in a mixture. Genomic DNA fragments are prepared by the treatment of genomic DNA with a restriction enzyme(e.g., Sau3AI) having a multiple restriction site in the genomic DNAs and DNase I, followed by an agarose gel electrophoresis and extraction of specific size of fragments(e.g., 25-500 bp). Recombinant DNAs are prepared by the insertion of a random oligonucleotide or a genomic DNA fragment to a specific site of a defective template, which includes the following two methods: One method is PCR-coupled recombination using the nucleotide sequence homology; and, the other method is sequence-directed recombination by ligase-aided direct ligation, which is independent on the nucleotide sequence homology. The PCR-coupled recombination imitates a natural recombination process occurring between homologous sequences in vivo, where PCR is carried out in a mixture of DNA fragments to be inserted and defective templates, to insert DNA fragments into defective genes through sequence homology. The sequence-directed recombination, regardless of sequence homology, directly inserts DNA fragments into a specific site of a defective template, by cleaving a specific site of a defective template with a restriction enzyme and ligating a DNA fragment to be inserted to the cleaved site of the defective template by using ligase. This recombination method employing a direct ligation is performed independent upon sequence homology, which assures the diversity of mutation.

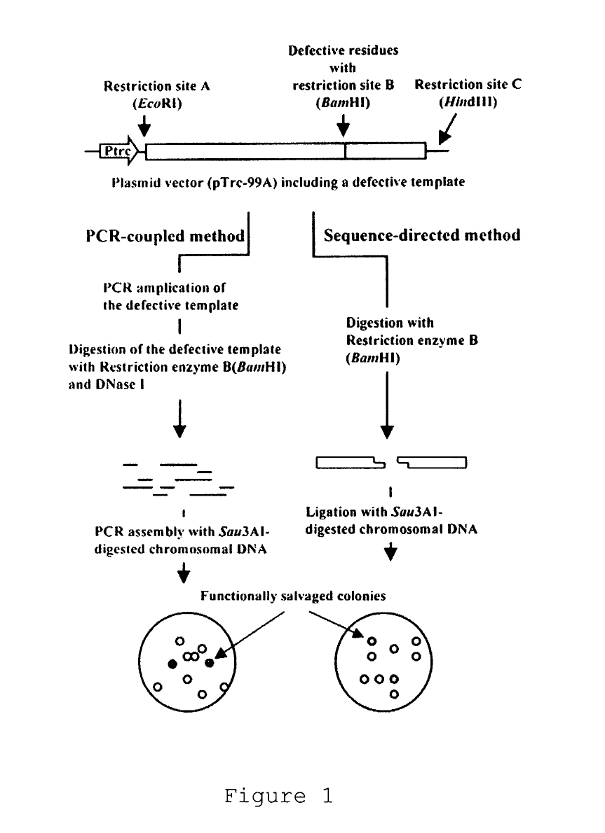

FIG. 1 shows a schematic diagram showing a method for manufacturing a mutant library of proteins with various sizes and sequences from a parental protein.

The present inventors prepared several defective genes, GFP∇176(+1), GFP∇176(+2), GFP∇172-3/176(+1), GFP∇172-3/176(+2), and GFP∇129-138/176(+2) by the deletion of nucleotide sequences encoding several amino acids of GFPuv which is a modified jellyfish GFP, and prepared the other defective gene, DHO∇68-70(+1) by the deletion of dozens of nucleotides in a gene coding for dihydroorotase. Then, they manufactured recombinant DNAs through the insertion of E. coli genomic DNA fragment into BamHI site of a defective gene by PCR-coupled recombination or sequence-directed recombination using a ligase, and constructed a library of microorganisms transformed with plasmids containing recombinant DNAs, and selected proteins with restored function of fluorescent protein or dihydroorotase activity from proteins expressed by the microorganisms. Amino acid sequence deduced from the E.coli genomic DNA fragment inserted into GFP ∇176(+2) includes SEQ ID NOs. 1, 2, 3, 4, 5, 6 and 7, and amino acid sequence deduced from the E.coli genomic DNA fragment into GFP ∇172-3/176(+2) includes SEQ ID NOs. 8, 9, 10, 11, 12, 13, 14, 15, 16 and 17. The present inventors selected Escherichia coli JM109/pMAL-c2/gfpS22transformed with a plasmid containing a recombinant DNA prepared by the insertion of E. coli genomic DNA fragment coding for an amino acid sequence represented SEQ ID NO. 4 to BamHI recognition site of a defective gene, GFP∇ 176(+2) and Escherichia coli JM109/pMAL-c2/gfpI5 transformed with a plasmid containing a recombinant DNA prepared by the insertion of E. coli genomic DNA fragment coding for an amino acid sequence represented as SEQ ID NO. 8 to BamHI recognition site of a defective gene, GFP∇172-3/176(+2) and deposited with the Korean Collection for Type Cultures(KCTC, #52, Oun-dong, Yusong-ku, Taejon 305-333, Republic of Korea), an international depository authority, as accession nos. KCTC 10059BP and KCTC 10058BP on Aug. 30, 2001, respectively.

The present invention is further illustrated in the following examples, which should not be taken to limit the scope of the invention.

### EXAMPLE 1

### Preparation of Fluorescent Proteins with Various Sizes and Sequences by PCR-coupled Recombination

Fluorescent proteins with various sizes and sequences were prepared by the expression of recombinant DNAs which was prepared by inserting restriction enzyme-treated genomic DNA fragments into a specific site of a defective gene coding for fluorescent protein by way of PCR-coupled recombination. First of all, two defective genes were constructed from a gene coding for a parental protein of GFPuv(Clontech, Canada), a modified jellyfish GFP as followings: One was constructed by the deletions of nucleotides coding for the 176^{th} amino acid on the 2^{nd} β-strand structure of GFPuv and an additional nucleotide as well, which was named as ‘GFP∇176(+1)’. The other was constructed by the deletions of nucleotides coding for the 176^{th} amino acid of GFPuv and additional two nucleotides as well, which was named as ‘GFP∇176(+2)’. In addition, deletions of nucleotides coding for the 172^{nd} and 173^{rd} amino acids of the GFPuv were made to the said two defective genes(i.e., GFP∇176(+1) and GFP∇176(+2)), were named as ‘GFP∇172-3/176(+1)’ and ‘GFP∇172-3/176(+2)’, respectively. Proteins expressed from these defective genes did not exhibit fluorescence.

Construction and amplification of the above defective genes are illustrated as followings: downstream N-terminal of a region to be deleted in a gene coding for a parental protein was amplified by employing a template of a gene coding for a parental protein and a pair of primers, i.e., a primer having nucleotide sequences encoding the N-terminal region of the parental protein and EcoRI recognition sequence and a primer having nucleotide sequences complementary to upstream of the region to be deleted and BamHI recognition sequence. Similarly, upstream C-terminal of a region to be deleted in a gene coding for a parental protein was amplified by employing a template of a gene coding for a parental protein and a pair of primers, i.e., a primer having nucleotide sequences complementary to the C-terminal region of the parental protein and HindIII recognition sequence and a primer having nucleotide sequences corresponding to downstream of the region to be deleted and BamHI recognition sequence. Then, amplified DNA for the downstream N-terminal and a plasmid, pTrc-99A were double-digested with EcoRI and BamHI, and were coupled by ligation using ligase. The amplified DNA for the upstream C-terminal part and the coupled plasmid were experienced a double-digestion with BamHI and HindIII followed by ligation to give a recombinant plasmid containing defective gene. The recombinant plasmid was then used for the amplification of the defective gene.

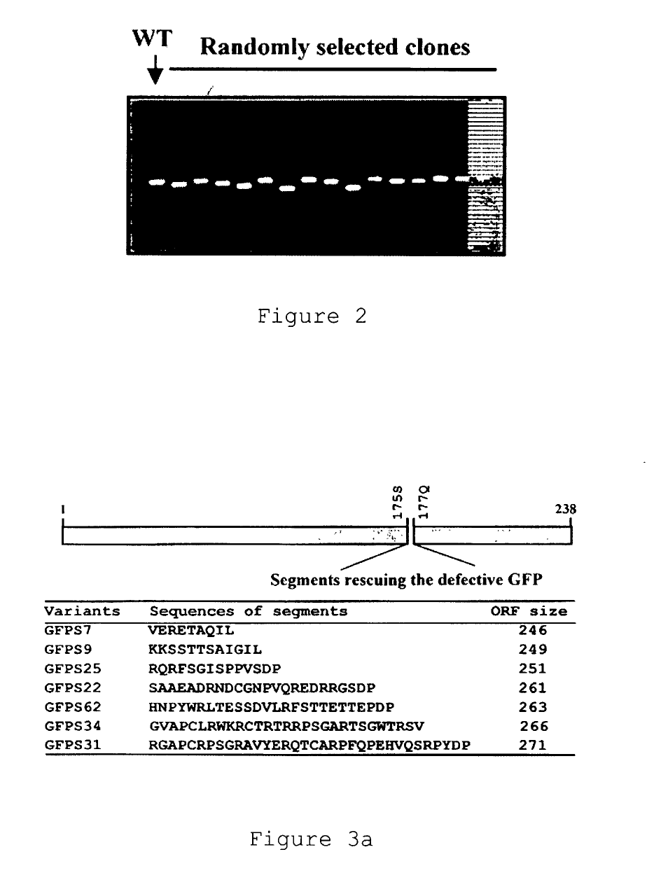

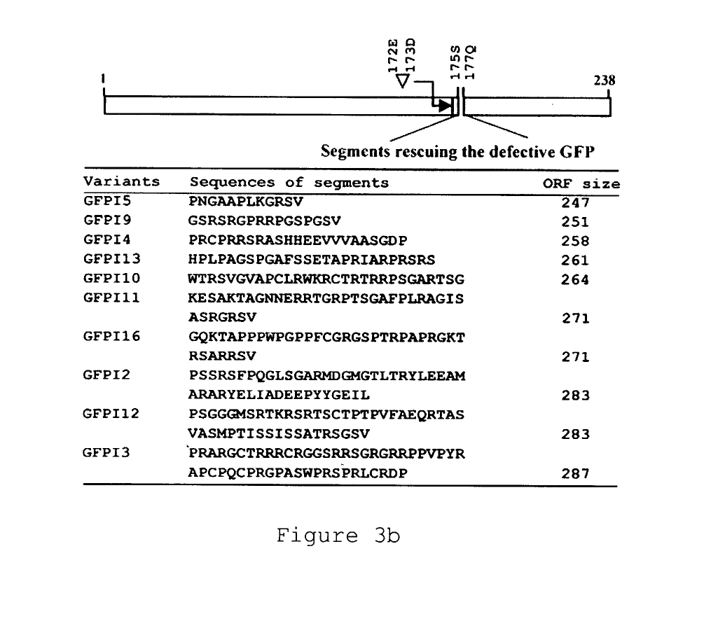

The amplified defective gene was digested with DNase I and subjected to agarose gel electrophoresis to extract DNA fragments of 50-150 bp in size. DNA fragments to be inserted were prepared by the digestion of E.coli genomic DNA with Sau3AI and DNase I followed by the extraction of 25-500 bp DNA fragments on the agarose gel. These randomly digested DNA fragments of defective gene and E. coli genomic DNA fragments were mixed and PCR was carried out to recombine and amplify. PCR was performed with 40 time repetitions of a cycle consisting of 1 min incubation at 94° C., 1 min incubation at 45° C., and 40 sec incubation at 72° C. The amplified recombinant products were cloned into a plasmid pTrc-99A and then used for the transformation of E. coli JM109 strain to construct a library of transformed microorganisms. FIG. 2 is a photograph showing the results of agarose gel electrophoresis of the genes of clones randomly selected from the library of microorganisms constructed with the defective template, GFP∇176(+2). As shown in FIG. 2, it was found that a library of microorganisms thus constructed contain genes of various sizes coding for fluorescent protein. The constructed microorganisms were illuminated with UV lamp to select clones exhibiting fluorescence by restoration of the fluorescent protein function. Nucleotide sequences were determined for the genes coding for the fluorescent proteins contained in the selected clones and their amino acid sequences were deduced from the nucleotide sequences. FIG. 3a represents amino acid sequences deduced from E. coli genomic DNA fragments inserted into the defective gene, GFP∇176(+2) and ORF sizes of recombinant DNAs thereof, respectively. The deduced amino acid sequences were represented as SEQ ID NOs: 1 to 7. As shown in FIG. 3a, the deduced amino acid sequences were varied in the size and sequence, indicating that the fluorescent proteins with various sizes and sequences can be produced by the method. Each of genes coding for the fluorescent proteins from the selected clones was fused to a gene coding for maltose binding protein, and the new recombinant genes were expressed in E. coli. The expressed proteins were purified, isolated and then examined with a fluorescence detector, indicating that the expressed proteins exhibited 2-16% fluorescence of the parental fluorescent protein.

Among the library of microorganisms constructed with the defective gene, GFP∇176(+2), Escherichia coli JM109/pMAL-c2/gfpS22 transformed with a plasmid containing a recombinant gene prepared by the insertion of E. coli genomic DNA fragment coding for an amino acid sequence(SEQ ID NO: 4) to BamHI recognition site of GFP∇176(+2) was deposited with the Korean Collection for Type Cultures(KCTC, #52, Oun-dong, Yusong-ku, Taejon 305-333, Republic of Korea), an international depository authority, as accession no. KCTC 10059BP on Aug. 30, 2001.

### EXAMPLE 2

### Preparation of Fluorescent Proteins with Various Sizes and Sequences by Sequence-directed Recombination Using a Ligase

Fluorescent proteins with various sizes and sequences were prepared by the expression of recombinant DNAs constructed by the direct ligation of E. coli genomic DNA fragment to a specific site of a defective gene for fluorescent protein by using ligase: each of defective genes constructed in Example 1 was cloned into a plasmid, pTrc-99A and the recombinant plasmid was linearized with BamHI restriction enzyme. E. coli genomic DNA was digested with Sau3AI, extracted fragments of 25-500 bp from the agarose gel, and used as DNA fragments to be inserted into the linearized plasmid. The 25-500 bp fragments were subjected to ligation to the linearized plasmids containing the defective gene. The resultant recombinant plasmids were used for the transformation of E. coli JM109 strain to construct a library of transformed microorganisms. The microorganisms constructed by the direct recombination with the defective gene, GFP∇ 172-3/176(+2) were illuminated with UV lamp and then clones exhibiting fluorescence were selected therefrom. Nucleotide sequences were determined for the genes coding for the fluorescent proteins contained in the selected clones and their amino acid sequences were deduced from the nucleotide sequences. FIG. 3b represents amino acid sequences deduced from E. coli genomic DNA fragments inserted into the defective gene, GFP∇172-3/176(+2) and ORF sizes of recombinant DNAs thereof, respectively. The deduced amino acid sequences were represented as SEQ ID NOs: 8 to 17. As shown in FIG. 3b, fluorescent proteins with various sizes and sequences were produced, similarly as in FIG. 3a. The fluorescent intensity were examined for the fluorescent proteins analogously as in Example 1, indicating that the fluorescent proteins exhibited 1-17% fluorescence of the parental protein.

Among the library of microorganisms constructed with the defective gene, GFP∇172-3/176(+2), Escherichia coli JM109/pMAL-c2/gfpI5 transformed with a plasmid containing a recombinant gene prepared by the insertion of E. coli genomic DNA fragment coding for an amino acid sequence(SEQ ID NO: 8) to BamHI recognition site of GFP∇172-3/176(+2) was deposited with the Korean Collection for Type Cultures, an international depository authority, as accession no. KCTC 10058BP on Aug. 30, 2001.

### EXAMPLE 3

### Preparation of Fluorescent Proteins from Genes Defected in Various Sites

Fluorescent proteins with various sizes and sequences were prepared by the expression of recombinant DNAs constructed by the insertion of restriction-enzyme treated E. coli genomic DNA fragments to two specific sites of a defective gene: Though genes defected in only one specific site were used in Examples 1 and 2, it would be possible to prepare fluorescent proteins of more various sizes and sequences by using the genes defected in two or more specific sites. In this regard, defective genes were constructed by mutating the green fluorescent protein in two or more sites, which are known to play an important role in exhibiting fluorescence or correct folding. Specifically, a new defective gene, GFP∇129-138/176(+2) was constructed by additional deletion of nucleotides coding for the 129^{th} to 138^{th} amino acids in the loop region of GFP from the defective gene, GFP∇176(+2) constructed previously. In a similar manner as in Example 1, the defective gene, GFP∇129-138/176(+2) was digested with DNase I and 50-150 bp fragments were isolated. Then, the extracted defective gene fragments and E. coli genomic DNA fragments were mixed and recombined and amplified by the technique of PCR. The amplified recombinant genes were cloned into plasmids and then used for the transformation of E. coli. Clones exhibiting green fluorescence were selected from the library of transformed E. coli.

### EXAMPLE 4

### Preparation of Proteins with Various Sizes and Sequences Having Dihydroorotase Activity

Dihydroorotase belongs to a cyclic amidohydrolase family along with hydantoinase, allantoinase and dihydropyrimidinase, and functions in the metabolism of purines and pyrimidines in vivo. Fluorescent proteins with various sizes and sequences having dihydroorotase activity were prepared by the expression of recombinant genes constructed by the insertion of restriction-enzyme treated E. coli genomic DNA fragments to a specific site of a defective dihydroorotase gene: A gene coding for the dihydroorotase was cloned from a derivative strain of E. coli K12 through PCR. Sequence homology and structure for various enzymes belonging to the cyclic amidohydrolase family were analyzed. Then, a defective dihydroorotase gene, DHO∇68-70(+1) was constructed by the deletion of 43 nucleotides ranging from 213 to 255. The proteins expressed from the defective gene did not exhibit a dihydroorotase activity. The defective gene was randomly digested, mixed with the E. coli genomic DNA fragments and recombined and amplified by the technique of PCR. The amplified genes were cloned into plasmids and transformed into E. coli X7014a strain defective in dihydroorotase activity followed by selection of the clones growing on a minimal medium, in a similar manner as in Example 1. The selected clones were examined for the dihydroorotase activity and the insertion sites of the genomic DNA fragments in defective gene were determined by nucleotide sequencing. Finally, the proteins with various sizes and sequences having dihydroorotase activity were prepared.

As clearly described and demonstrated above, the present invention provides a method for manufacturing a mutant library of proteins with various sizes and sequences from a parental protein, microorganisms transformed with plasmids containing recombinant DNAs prepared by the insertion of a genomic DNA fragment into a defective template, a process for preparing proteins with different sizes and sequences from the parental protein which comprises the steps of culturing the transformed microorganisms and obtaining desired proteins from the culture, and proteins prepared by the said process. In accordance with the invention, a mutant library of proteins with various sizes and sequences can be manufactured from a parental protein in an efficient and simple manner, by constructing a library of microorganisms transformed with recombinant plasmids containing E. coli genomic DNA fragments inserted into defective genes and selecting clones expressing proteins with restored functions or modified characteristics. The proteins produced by the invention can be further improved in terms of stability, activity, substrate specificity, etc. by way of protein engineering, which makes possible its wide application in the development of biological materials such as antigens and antibodies for clinical use and the improvement of industrial enzymes.

What is claimed is:

1. A method for manufacturing a mutant library of proteins with various sizes and sequences from a parental protein which comprises the steps of:
(i) artificial modification into a gene coding for a parental protein to give a defective template;
(ii) construction of a library of microorganisms transformed with plasmids containing recombinant DNAs prepared by the insertion of a random oligonucleotide or a restriction enzyme-treated genomic DNA fragment to the defective template; and,
(iii) selection of proteins having restored function or changed characteristics from proteins expressed by the library of microorganisms transformed with plasmids.

2. The method for manufacturing a mutant library of proteins with various sizes and sequences from a parental protein of claim 1, wherein the artificial modification includes deletion of nucleotides coding for several amino acids or a domain at a specific site of a gene coding for the parental protein, frame shift or combination thereof.

3. The method for manufacturing a mutant library of proteins with various sizes and sequences from a parental protein of claim 1, wherein the insertion of a random oligonucleotide or a restriction enzyme-treated genomic DNA fragment to a defective template is carried out by PCR-coupled recombination.

4. The method for manufacturing a mutant library of proteins with various sizes and sequences from a parental protein of claim 1, wherein the insertion of a random oligonucleotide or a restriction enzyme-treated genomic DNA fragment to a defective template is carried out by sequence-directed recombination employing a ligase.

5. A microorganism transformed with a plasmid containing a recombinant gene prepared by the insertion of E. coli genomic DNA fragment to BamHI recognition site of GFP∇ 176(+2), a defective gene of GFPuv.

6. The microorganism of claim 5, wherein the amino acid sequence deduced from E.coli genomic DNA fragment is SEQ ID NOs 1, 2, 3, 4, 5, 6 or 7.

7. Escherichia coli JM109/pMAL-c2/gfpS22(KCTC 10059BP) transformed with a plasmid containing a recombinant gene prepared by the insertion of E. coli genomic DNA fragment coding for an amino acid sequence(SEQ ID NO: 4) to BamHI recognition site of GFP∇ 176(+2), a defective gene of GFPuv.

8. A fluorescent protein selected from proteins expressed by the microorganism of claim 5.

9. A microorganism transformed with a plasmid containing a recombinant gene prepared by the insertion of E. coli genomic DNA fragment to BamHI recognition site of GFP∇172-3/176(+2), a defective gene of GFPuv.

10. The microorganism of claim 9, wherein the amino acid sequence deduced from E.coli genomic DNA fragment into GFP 172-3/176(+2) is SEQ ID NOs 8, 9, 10, 11, 12, 13, 14, 15, 16 or 17.

11. Escherichia coli JM109/pMAL-c2/gfpI5(KCTC 10058BP) transformed with a plasmid containing a recombinant gene prepared by the insertion of E. coli genomic DNA fragment coding for an amino acid sequence(SEQ ID NO: 8) to BamHI recognition site of GFP∇172-3/176(+2), a defective gene of GFPuv.

12. A fluorescent protein selected from proteins expressed by the microorganism of claim 9.

13. A microorganism transformed with a plasmid containing a recombinant gene prepared by the insertion of E. coli genomic DNA fragment to BamHI recognition site of DHO∇68-70(+1), a defective gene of dihydroorotase.

14. A protein having dihydroorotase activity which is selected from proteins expressed by the microorganism of claim 13.

15. A method for manufacturing a mutant library of proteins with various sizes and sequences from a parental protein which comprises the steps of culturing Escherichia coli JM109/pMAL-c2/gfpS22(KCTC 10059BP) of claim 7 and obtaining desired proteins from the culture.

16. A method for manufacturing a mutant library of proteins with various sizes and sequences from a parental protein which comprises the steps of culturing Escherichia coli JM109/pMAL-c2/gfpI5(KCTC 10058BP) of claim 11 and obtaining desired proteins from the culture.
